(19) United States
(12) Patent Application Publication
(10) Pub. No.: US 2017/0027664 A1
(43) Pub. Date: Feb. 2, 2017

(54) Device for Reducing Effect and Symptomologies of Bruxism and/or Morsicatio Buccarum

(71) Applicant: John B. Dority, Comer, GA (US)

(72) Inventor: John B. Dority, Comer, GA (US)

(21) Appl. No.: 15/216,794

(22) Filed: Jul. 22, 2016

Related U.S. Application Data
(60) Provisional application No. 62/199,328, filed on Jul. 31, 2015.

(51) Int. Cl. A61C 5/14; A61M 31/00

(52) U.S. Cl. CPC A61C 5/14; A61M 31/00; A61F 2005/563

(57) ABSTRACT

The present invention is a device for reducing or minimizing the symptoms and/or effects of bruxism and/or morsicatio buccarum comprising a single-piece main body having an approximately U-shaped cross section that fits over one or more teeth. The body has an upper surface, a lower surface and a descending skirt one on either side of said main body and parallel to one another. Each of the descending skirts has an interior side, an exterior side, an top portion and a bottom portion. The top portion is integral to the side of the main body and is narrower in width then the bottom portion. The device is affixed to the tooth or teeth through the compression force produced by and between the descending skirts.

### CROSS-REFERENCE TO RELATED APPLICATIONS

Not applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates to the field of dental and oral care, specifically, to devices and methods for preventing or reducing unintentional clenching or grinding of teeth also referred to as bruxism and preventing or reducing damage to the inside of the cheek resulting from chewing or biting also referred to as morsicatio buccarum.

### BACKGROUND OF THE INVENTION

Morsicatio buccarum, also termed chronic cheek biting and chronic cheek chewing, is a condition characterized by chronic irritation or injury to the buccal mucosa caused by repetitive chewing, biting, or nibbling and by definition is unrelated to the normal function of eating or talking.

Morsicatio buccarum is fairly common, with one in every 800 adults showing evidence or active lesions at any one time. It is more common in people who are experiencing stress or psychological conditions. The prevalence in females is double the prevalence in males, and it is two or three times more prevalent in people over the age of thirty five. Although lesions which may be caused by morsicatio buccarum are usually harmless, repeated biting of the buccal mucosa can lead to oral trauma such as mouth sores and ulcers, increasing discomfort and pain which may accompany the condition. Injury to the buccal mucosa may cause a raised surface which is prone to repeated biting, chewing, or nibbling which may further exasperate the condition and increase associated pain.

Bruxism is the excessive grinding of the teeth and/or excessive clenching of the jaw and by definition is unrelated to the normal function of eating or talking. Bruxism is a common problem with a reported prevalence ranging from 8-31% in the general population. Bruxism can cause such minor symptoms that the individual may not be aware of the condition, which could account for the lower estimated prevalence rate. Some of the symptoms associated with bruxism include: excessive tooth wear, particularly attrition, which flattens the biting surface; tooth fractures and repeated failure of dental restorations such as fillings, crowns and the like; hypersensitive teeth caused by wearing away of the thickness of insulating layers of dentin and enamel around the dental pulp; inflammation of the periodontal ligament of teeth, which may make them sensitive when biting, and possibly loosen the teeth; cheek or lip biting; indentations of the teeth in the tongue also referred to as “crenated tongue” or “scalloped tongue”; hypertrophy of the mastication muscles; tenderness, pain or fatigue of the mastication muscles during chewing or other jaw movement; trismus which is a restriction of the mouth opening; pain or tenderness of the temporomandibular joints, which may manifest as preauricular pain, or otalgia; clicking of the temporomandibular joints; and headaches, particularly pain in the temples, caused by muscle pain associated with the temporalis muscle.

There are two main types of bruxism. One which occurs during sleep referred to as sleep bruxism and the other which occurs during wakefulness or awake bruxism. Dental damage may be similar in both types, but the symptoms of sleep bruxism tend to be worst on waking and improve during the course of the day, and the symptoms of awake bruxism may not be present at all on waking, and then worsen over the day. The causes of bruxism are not completely understood, but probably involve multiple factors. Awake bruxism is thought to have different causes than sleep bruxism, and is more common in females, whereas males and females are affected in equal proportions by sleep bruxism. Several treatments are in use, although there is little evidence of robust efficacy for any particular treatment.

A number of dental guards and occlusal splints have been developed for reducing the effects of bruxism including, U.S. Pat. Nos. 4,044,762, 4,063,552, 4,765,324, 4,955,393, 5,031,638, 5,406,963, 5,339,832, 5,293,880, 6,036,487, 5,931,164, 5,566,684, 6,109,266, 6,082,363, 6,986,354, 6,935,857, 6,932,088, 6,584,978, 7,549,423, 7,299,804, 8,100,131, 8,539,955 and 8,607,798. As an example of these types of mouth guards, U.S. Pat. No. 5,876,199 is directed to an orthodontic appliance comprising a U-shaped labial-buccal flange, a U-shaped lingual flange, an isthmus connecting the labial-buccal and lingual flanges and a tooth receiving trough sized to act as a sole means for retaining the appliance in place during use. This appliance is designed to envelop all of the teeth of the upper jaw or maxilla. Some commercially available dental guards of this configuration, U.S. Pat. No. 7,328,706, may be adapted to fit securely to the configuration of the teeth of the user. This can be done by using heat softening polymer that when cooled retains the impression of the teeth impressed into the guard after heating.

Unfortunately, these devices are bulky and deform the lips when placed on the teeth making them uncomfortable during use.

Another U.S. patent, U.S. Pat. No. 8,733,364, is directed to a bruxism treatment device comprising a U-shaped elongated flexible band having planar bite pads on the ends. The band is fitted between the base of the teeth and the lip along the gum line with the ends rising up and extending over the rear molars forming the bite pads. This device is designed for placement in the lower jaw or mandible. U.S. Pat. No. 6,978,786 has a similar configuration replacing the bite pads with fluid filled pads connected to each other to allow fluid to be exchanged between the pads. This guard is designed to treat dysfunctions of the mandibular joint caused by one-sided loading. The application of pressure to one pad causes increased fluid in the other equalizing the pressure to both mandibular joints thereby relieving the symptoms of mandibular joint dysfunction. U.S. Pat. No. 8,689,796 has a similar configuration to U.S. Pat. No. 8,733,364 wherein the bite pads have been replaces with troughs that fit over the molars. Like the dental guard and occlusal splints described above these devices are also bulky and can be uncomfortable during use.

U.S. patent application, U.S.2007/0079833 is directed to an intraoral mandibular device having a main body means for attachment to the roof of mouth, retention means extending from the main body to maintain the device in place during use and a protrusive element descending from the central portion of the main body. The main body is designed to conform to the roof of the mouth of each individual user and provides wire clip in either side of the main body that fits into the gap between the molars holding the device in place. In a similar device this retention means can be a single wire attached by its ends to either side of the main body and fitted across the front teeth. These devices are custom made providing the exact inverse impression of the roof of the user's mouth so that the device is comfortable during use. Unfortunately, this increases the cost substantially. In addition, the wire clips or retaining wire press against the teeth and can cause discomfort resulting from sore teeth at the wire contact locations and headaches if the pressure is severe.

Other devices are similarly constructed as the full mouth guard configurations but are designed to be fitted only on the incisors and canines. These include U.S. Pat. No. 7,571,727 and U.S. patent applications U.S.2014/0109919 and U.S. 2014/0190491. These devices usually require an protrusive element distending from the central portion so that when the front teeth contact his element is provides a sufficient gap in the rear teeth to prevent contact. Unfortunately, the gap between the front teeth on the mandible and the front teeth on the maxilla is different for every individual consequently these devices may be appropriate from some but not others. In addition, individual suffering from an underbite, a condition in which the lower teeth overlap the upper teeth resulting from mandibular underdevelopment or to excessive maxillary development, would not be able to use these devices.

Consequently, there is a need for a device for treating the effects of bruxism and/or morsicatio buccarum that is not bulky or uncomfortable to were increasing patient compliance, that does not exert pressure on both sides of the upper or lower jaw to maintain the mouth guard in place and can be used for all individuals independent of the extend of the gap between the front teeth or in cases where the user has an underbite.

### SUMMARY OF THE INVENTION

The present invention is a device for reducing or minimizing the symptoms and/or effects of bruxism and/or morsicatio buccarum comprising a single-piece main body having an approximately U-shaped cross section that fits over one or more teeth. The body has an upper surface, a lower surface and a descending skirt one on either side of said main body and parallel to one another. Each of the descending skirts has an interior side, an exterior side, an top portion and a bottom portion. The top portion is integral to the side of the main body and is narrower in width then the bottom portion.

In one embodiment, the device is affixed to the tooth or teeth through the compression force produced by and between the descending skirts. The distance between the upper surface and the lower surface of the main body ranges from approximately 2 mm to approximately 10 mm, 3 mm to approximately 8 mm or 4 mm to approximately 6 mm. The main body may cover one or more teeth and may be retained in place utilizing the spacing between teeth. For example, a wire or string may be positioned between the bottom portions of the descending skirts that fit within the gap between the teeth and made of a high tensile strength break resistant polymer. Alternatively, a rib may be formed in the lower surface of the single-piece main body that nests within the space between the teeth.

The device may be made of a polymer and may be constructed of a heat formable plastic and/or a plastic that may be embedded with a flavoring or an agent such as a saliva inducer, a teeth whitener or a medicine and may be disposable.

Another aspect of the invention is a method of treating the symptomologies of bruxism and/or morsicatio buccarum comprising the steps of affixing at least one of the devices disclosed above to at least one tooth during a time when bruxism and/or morsicatio buccarum occurs. The tooth or teeth may be premolars or molars on the lower jaw or maxilla.

### DESCRIPTION OF THE FIGURES

FIG. 1: (A) is a perspective view of the device in place on a single tooth, (B) is a frontal perspective view of a simple U-shaped device of the present invention, (C) is a frontal perspective view of the device having clasping projections on the ends of the depending skirts and a convex upper surface, (D) is a frontal perspective view of the device having a wire or string retaining means, (E) is a frontal perspective view of the device having a rib retaining means, (F) is a base perspective view of the device having a rib retaining means and (G) is a frontal perspective view of a simple U-shaped device of the present invention with rounded corners.

### DETAILED DESCRIPTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

The phrase “means for retaining” as used herein refers to an element of the device that acts to retain the device in place during use. The means utilizes the gap provided between the teeth by providing a space filling ridge or rib on either side of the device that fits into the gaps provided on both sides of the teeth securely holding the device in place. Alternatively, the means may utilize the spacing between the teeth by providing a wire or string between the sides of the device that is pressed into this space during use holding the device in place.

The term “about” or “approximately” as used herein refer to an numerical value, amount or magnitude that may vary in a range from 1% to 15% and preferably from 5% to 10% or any specific percentage in either range.

The present invention is a device for reducing or minimizing the symptoms and/or effects of bruxism and/or morsicatio buccarum comprising a single-piece main body having an approximately U-shaped cross section that fits over one or more teeth. The body has an upper surface, a lower surface and a descending skirt one on either side of said main body and parallel to one another. Each of the descending skirts has an interior side, an exterior side, an top portion and a bottom portion. The top portion is integral to the side of the main body and is narrower in width then the bottom portion.

### I. Main Body

The main body 2 of the device 10 provides a biting surface of a thickness that keeps the teeth in the maxilla and mandible from contacting each other when the mouth is closed. The main body 2 may be provided in a variety of shapes that provide a platform for the tooth or teeth contacting the device 10 during use and maintains the general alignment of the teeth in the upper and lower jaws. This surface may have the length of the one or more teeth it is intended to cover and may range from about 5 mm to about 40 mm. The width of the main body 2 may be slightly larger than the width of a tooth to about 2.0 times that width. In one embodiment, the width is only slightly larger than the width of the tooth or teeth the device is intended to cover so that it does not significantly impact the tongue and is comfortable during use.

The main body 2 may be constructed from a variety of polymeric materials that maintain structural integrity when compressed and flexibility over continued use. It may be prepared as a single piece or assembled from multiple pieces. If the main body 2 is prepared as a single piece, then it will be constructed of a material that ensures the descending skirts 4 on either side will maintain sufficient rigidity to hold the device 10 in place during use. If the main body 2 is assembled from multiple pieces, then the upper and lower surfaces may be prepared from a more flexible material to reduce stress on the teeth during contact while the internal structural element connecting the descending skirts 4 is prepared from a more rigid material that maintains the device in place during use. In a disposable configuration, the main body 2 may be detached from the internal structural element (i.e., scaffold), discarded and replaced with a new main body 2.

### II. Descending Skirts

Descending skirts 4 are provided on either side of the main body 2 to bracket the tooth or teeth and secure the device 10 in place during use. In one embodiment the tooth or teeth to which the device 10 is affixed are molar and/or premolars. Most molars and/or premolars are wider at the crown (i.e., the contact surface) than at the base. Consequently, the descending skirts 4 may be constructed to exert force about the crown or base of the tooth or teeth.

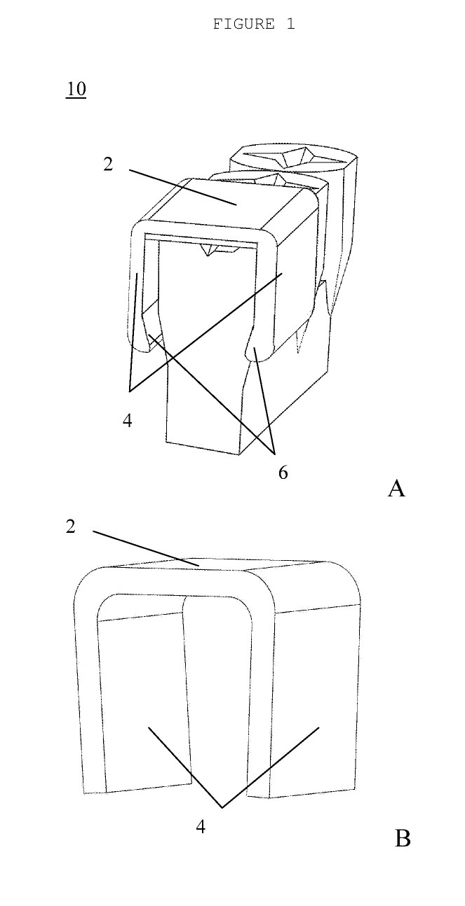

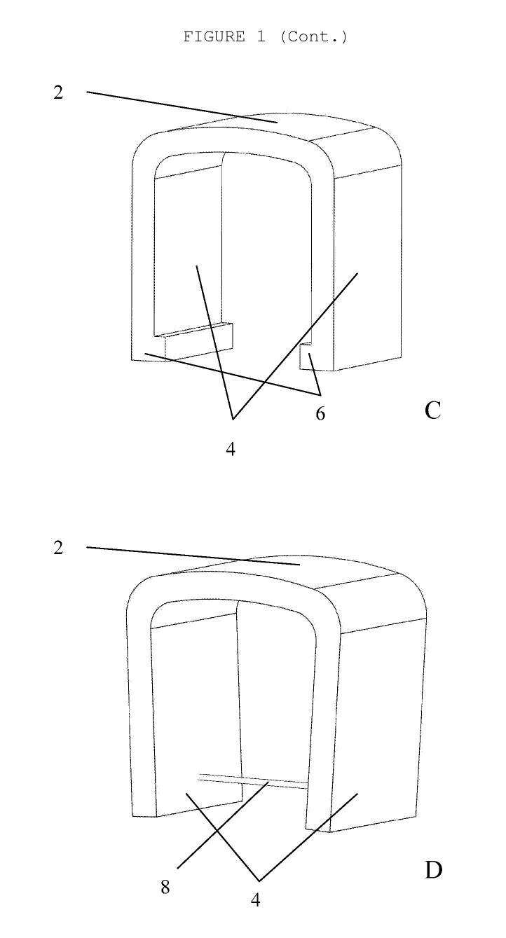

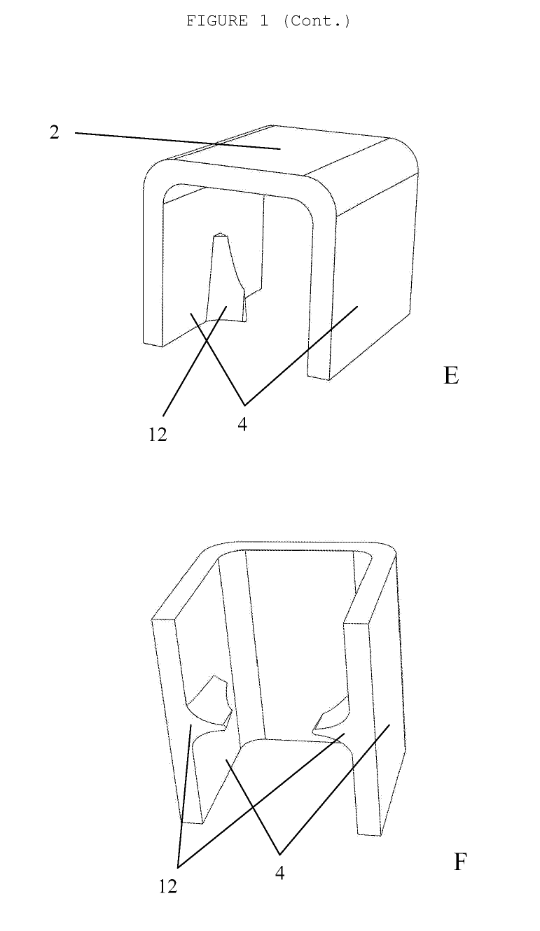

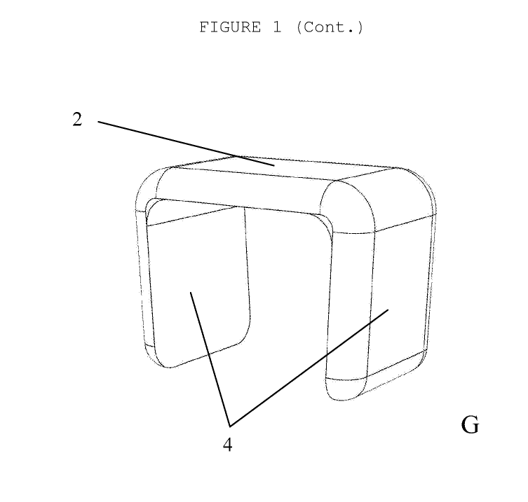

In one embodiment, the interior surfaces of the descending skirts 4 are planar. In this configuration (see FIG. 1B), the descending skirts 4 are not parallel but angled inward such that the upper portions of the skirts 4 affixed to the main body 2 are farther apart than their lower portions.

This design creates a spring force between the skirts 4 that that exerts pressure on both sides of the tooth or teeth holding the device 10 in place.

In another embodiment (see FIG. 1C), the lower portion of the descending skirts 4 are provided with a ridge that extend along the edge of the lower portion projecting inwardly toward each other. Alternatively (see FIG. 1A), the internal surface of the descending skirts 4 (i.e., the surface facing the tooth or teeth) are tapered forming a ridge at the base of the lower portions of the descending skirts 4. This design acts like a clamp or jaw maintaining the device in place during use.

The length (i.e., the distance the descending skirt 4 extends from the main body) of the descending skirts 4 may vary depending on the side of the tooth or teeth the device is applied. Consequently, the length can range from about 5 mm to about 15 mm. The width (i.e., the distance from one side to the other side of the descending skirt 4) will depend on the number of teeth to be covered by the device 10. Consequently, the width may range from about 5 mm to about 15 mm to cover a single tooth or from about 15 mm to about 40 mm to cover more than one tooth. The width may also be the same width as, a smaller width than, or a greater width than the width of the main body 2.

The descending skirt 4 may be made from a variety of polymers and may be made of the same or different polymers as the main body 2. If the descending skirts 4 are made of the same polymer as the main body 2 the device 10 may be made as a single piece. Alternatively, if the descending skirt 4 is made of a different material, then it is preferable that the material used be a semi-rigid polymer that may flex enabling the device 10 to be affixed securely to the tooth or teeth during use. In this construction, it is preferable that the descending skirts 4 are connected through a scaffold that provides a platform to receive and secure the main body 2. The scaffold may be integral to the upper portions and connecting the two depending skirts 4. Alternatively, this scaffold made be provided separately having a top portion for receiving and securing the main body 2 and connectors on opposite ends for receiving and securing the descending skirts 4. The scaffold may be made of the same material or different material as the descending skirts 4. In one embodiment the scaffold is made of dental steel.

### III. Means for Retaining

A variety of methods may be utilized to secure the device 10 in place during use including a tension fitting (described above, see FIG. 1A-C), a retaining wire or string 8 (see FIG. 1D) or a retaining rib 12 (see FIG. 1E). When utilizing the wire 8 or rib 12 configuration, the device 10 will be secured on at least two teeth and depending on the number of teeth one or more wires 8 or ribs 12 may be utilized. For example, if the device is designed to cover three teeth, then it may provide as many as two wires 8 or ribs 12. In addition, each of these methods may be used in conjunction with any or all of the other methods. For example, the device may be constructed to cover three molars utilizing tension between the descending skirts 4 with two sets of ribs 12 and having one string 8 extending between and from the peak of one set of ribs 12.

In the retaining wire configuration a wire or sting 8 is provided extending between and about the middle of the interior surface at the lower portion of the descending skirt 4. If the retaining wire 8 is made of metal it is preferably made of dental steel and in a thickness that will easily fit between the spacing between teeth. If the retaining wire is a string 8 it is preferably made of a material used to prepare dental floss.

### IV. Imbedded Polymer

In one embodiment, the device may be made of a polymer in which medications, flavorings or other agents may be embedded. This polymer may be used to prepare the entire device 10 or certain elements separately such as the main body 2 and/or the descending skirts 4. For example, polymers such as those produced; manufactured and sold by Add The Flavor <http://www.addtheflavor.com/Technology> may be utilized.

### Assembly

The present invention may be prepared as a single piece or constructed of two or more pieces. If prepared as a single piece no assembly will be required and the device may be used immediately after being removed from its packaging. When the device is made of more than one piece it may be provided fully assembled and ready for use or some assembly may be required. For example, the descending skirts affixed to the scaffold may be provided along with a number of main bodies. The additional bodies may be provided as single use attachments to the scaffold of the descending skirts assembly or they may be made of a polymer embedded with different agents including flavorings and medications. The medications include agents that reduce bacteria in the mouth, to freshen breath or to reduce acidity in the saliva. The main body may be selected based on the desires of the user.

USE The device is unpackaged and assembled if needed. If the device is provided in the configuration shown in FIGS. 1A-C the opening between the descending skirts is positioned on the inside corner of the tooth or teeth the device is intended to cover. The device is then pressed onto the tooth or teeth thereby widening the opening between the descending skirts until both skirts envelope the crown of the tooth or teeth. Once this occurs, the device clips securely onto the tooth or teeth.

If the device is one shown in FIGS. 1D-E the opening of the descending skirt is positioned in the inside corner of the teeth with the wire, string or rib positioned over the area between the teeth. The device is then pressed onto the teeth thereby widening the opening between the descending skirts until both skirts envelope the crown of the tooth or teeth and the wire, string or rib slipping into the gap between the teeth. Once this occurs, the device clips securely onto the tooth or teeth and is further held in position by the wire, string or ribs.

Removal may be performed by grasping the ends of the descending skirts and lifting upward or by grasping the ends of the main body and lifting upward.

I claim:

1. A device for reducing or minimizing the symptoms and/or effects of bruxism and/or morsicatio buccarum comprising:
a single-piece main body having an approximately U-shaped cross section, said body having an upper surface, a lower surface and a descending skirt one on either side of said main body and about parallel to each other wherein each of said descending skirts have an interior side, an exterior side, an top portion and a bottom portion wherein said top portion is integral to said side of said main body and the distance between the top portion of said descending skirts is equal to or wider in width then said bottom portion.

2. The device according to claim 1, wherein the distance between said upper surface and said lower surface of said main body ranges from approximately 1 mm to approximately 10 mm.

3. The device according to claim 1, wherein the distance between said upper surface and said lower surface of said main body ranges from approximately 3 mm to approximately 8 mm.

4. The device according to claim 1, wherein the distance between said upper surface and said lower surface of said main body ranges from approximately 4 mm to approximately 6 mm.

5. The device according to claim 1, wherein said single-piece main body covers at least one tooth.

6. The device according to claim 1, wherein said single-piece main body covers at least two teeth.

7. The device according to claim 1, further comprising a means for retaining said device in place utilizing the spacing between teeth.

8. The device according to claim 7, wherein said means for retaining is a wire or string wherein said wire or string is positioned between said bottom portions of said descending skirts.

9. The device according to claim 8 wherein said string is made of a high tensile strength break resistant polymer.

10. The device according to claim 7, wherein said means for retaining is a rib formed in said lower surface of said single-piece main body that fits within the space between the teeth.

11. The device according to claim 1, wherein said device is made of polymer.

12. The device according to claim 11, wherein said polymer is a heat formable plastic.

13. The device according to claim 11, wherein said polymer is imbedded with a medicament or flavoring.

14. The device according to claim 13, wherein said medicament is a saliva inducer, a teeth whitener or a medicine.

15. The device according to claim 1, wherein said device is affixed to at least one tooth through the compression force produced by and between said descending skirts.

16. The device according to claim 1, wherein said device is disposable.

17. A method of treating the symptomologies of bruxism and/or morsicatio buccarum comprising the steps of affixing at least one device comprising single-piece main body approximately rectangular in shape having an upper surface, a lower surface and a descending skirt one on either side of said main body and about parallel to each other wherein each of said descending skirts have an interior side, an exterior side, an top portion and a bottom portion wherein top portion is integral to said side of said main body, wherein the distance between said top portion of said descending skirts being equal to or wider in width then said bottom portion to at least one tooth during a time when bruxism and/or morsicatio buccarum occurs.

18. The method according to claim 15, wherein said single-piece device is affixed to at least one tooth during a time when bruxism and/or morsicatio buccarum occurs.

19. The method of claim 15, wherein said tooth or said teeth are premolars or molars.

20. The method according to claim 15, wherein said at least one tooth is on the lower jaw or the maxilla.
